(19) United States
(12) Patent Application Publication
(10) Pub. No.: US 2014/0349957 A1
(43) Pub. Date: Nov. 27, 2014

(54) Compositions for Photodynamic Therapy Chemically Modified to Increase Epithelia Penetration and Cellular Bioavailability

(71) Applicant: YAGNA LIMITED, London (GB)

(72) Inventor: Giussepe Trigiante, Watford (GB)

(21) Appl. No.: 14/362,671

(22) PCT Filed: Dec. 7, 2012
(86) PCT No.: PCT/IB12/02794

§ 371 Date: Jun. 4, 2014

Related U.S. Application Data
(60) Provisional application No. 61/568,028, filed on Dec. 7, 2011.

(51) Int. Cl. A61K 41/00

(52) U.S. Cl. CPC A61K 41/0057
USPC 514/49

(57) ABSTRACT

The present invention describes a photodynamic prodrug, i.e., a substituted 4-thiothymidine (4-TT), which is able to cross the body's epithelia tissues such as the skin, oral cavity, nasal cavity, pulmonary tract, digestive tract, and blood-brain barrier, including the use of such a prodrug in a topical application for the treatment of skin hyperplasias, including skin cancer, psoriasis, keloids, actinic keratosis, and the like.

### CROSS REFERENCE TO RELATED APPLICATION

This application claims benefit under 35 U.S.C. §119(e) to U.S. Provisional Application No. 61/568,028, filed Dec. 7, 2011, which is incorporated by reference herein in its entirety.

### BACKGROUND OF THE INVENTION

1. Field of the Invention

The present invention relates generally to cell permeability and photodynamic therapy, and more specifically to a photodynamic therapy molecule, 4-thiothymidine, chemically modified into a prodrug able to cross the body's epithelia tissues such as the skin, oral cavity, nasal cavity, pulmonary tract, digestive tract, and blood-brain barrier, including the use of such a molecule in a topical application for the treatment of skin hyperplasias, including skin cancer, psoriasis, keloids, actinic keratosis, and the like.

2. Background Information

Epithelial hyperplasias are among the most common cell proliferation disorders. They all involve excessive proliferation of a subset of cells in the lining of an organ or in the membrane which constituted the interface between the body and the outside. Their severity can range from mild in the case of skin psoriasis or actinic keratosis (AK), to serious in the case of epithelial cancers (carcinomas) such as basal cell carcinoma (BCC), squamous cell carcinoma (SCC), melanoma (skin), head and neck cancer, stomach cancer, intestinal cancer, and bladder cancer.

Skin cancers in their various forms account for the most frequent cancers. Only one of them, melanoma, is seriously life threatening. Non-melanoma cancers such as BCC although very common are relatively benign; SCC are intermediate in danger because they can occasionally metastasize. Hyperplasias such as actinic keratosis (AK) are so called precancerous lesions because they can lead to SCC if left untreated.

Apart from these, there are other conditions that are not life threatening but are the cause of much distress for the patient and require treatment. Psoriasis is an autoimmune disease which results in chronic inflammation of patches of skin causing itching and pain.

Keloids are instead abnormal scars which grow to many times the size of the original wound on susceptible individuals. The main treatment is surgical removal but this unavoidably results in another wound with a 50% chance of the keloid returning. A non invasive treatment would be most needed.

Photodynamic therapy (PDT) is a novel treatment for hyperproliferative diseases of the skin and internal epithelia. It involves the administration, topically or systemically, of a photosensitive agent which will ideally concentrate in the proliferating tissues of the body. The compound itself is inactive but upon irradiation with a light of a specific wavelength the molecule is chemically activated and stimulated to undergo chemical reactions which either damage the cell directly or result in the production of species which is, in turn, noxious to the cells. This way the chemotherapeutic action is physically confined to an area of interest instead of extending to the whole body of the patient with unpleasant and harmful side effects. The field of applicability of PDT is naturally limited by the accessibility of tissue to the light source.

Internal cancers such as lung, bladder, and those of the digestive tract (e.g., stomach/colon) both represent major causes of mortality and a significant percentage of all cancer deaths. Even though modern preventive approaches have succeeded in reducing incidence, on the therapy side little has been done in terms of specificity of treatment, i.e., non-chemotherapic approaches. These cancers all present an interface to air, which makes them potentially accessible to a light emitting probe and therefore to PDT.

The main players in the PDT field today are porphymer sodium (PHOTOPRIN™) and 5 amino levulinic acid (ALA). PHOTOPRIN is a porphyrin derivative which has been licensed for systemic use in the US and the EU for the treatment of bronchial, lung, bladder and esophageal cancer. ALA instead is a porphyrin precursor which is converted into protoporphyrin IX directly in cells; it is administered topically and it is licensed for the treatment of actinic keratosis. Its mode of administration involves applying the emulsion on the affected area, then following 14 hours irradiate with red light. An ALA derivative, methyl aminolevulinate (MAL) has been developed and under the trade name METVIX™ is in use for pre-malignant conditions of the skin (BCC, AK).

The main issue with topical delivery of drugs is poor barrier penetration. All human epithelia have some kind of protective barrier function, because of the boundary role played against the outside environment. This imposes the requisite of impermeability to, for example, bacteria or viruses or toxic chemicals and the need to retain water inside. The most important epithelium for pharmaceutical purposes is the skin, whose structure is outlined in FIG. 1. The outermost layer of the skin is called the stratum corneum or cornified layer. It is a very compact tissue of dead cells crosslinked by keratin proteins and replete with fatty acids and esters, and it is thus the most effective biological barrier in the body, able to prevent our dehydration and shut out infectious agents. Other relevant epithelia are the oral and gut mucosa and the bronchial mucosa. They are more permeable than the skin because they are designed to absorb and secrete liquids, gases, and/or nutrients but still provide a formidable barrier function by means of their cellular tight junctions which expose to the candidate drug a quasi-continuous layer of hydrophobic, cellular membrane phospholipids. This membrane is also the last step in the pharmacokinetics of any drug, as the penetration into the target cell is necessary in order to achieve pharmaceutical activity. Finally, the blood-brain barrier is a very challenging epithelium separating the circulation from the brain tissues which behaves to all effects as a highly hydrophobic lipid sheet, thus preventing delivery of highly desirable neuroactive drugs to the central nervous system (CNS).

Many methods have been devised to overcome these significant obstacles. The use of vehicles called penetration enhancers, mixed with the drug, allows improvement in the extent of skin penetration; there are many such enhancers known to the skilled artisan. However, enhancers do not remain with the drug beyond the initial application site as they are chemically separated molecules and therefore are not effective at increasing penetration through any of the barriers following the first (e.g. the cell and blood-brain barrier).

For this reason another method has been attempted with relative success which is the direct chemical derivatization of drugs with groups intended to change the drug hydrophobicity and allowing a better pharmacokinetic distribution. They behave to all effects as chemically attached enhancers. This strategy will allow the drug penetration through all the membranes along its path to the active site; it is essential, however, that the conjugated moieties be removed from the drug molecule following delivery to said site or else its mechanism of action (pharmacodynamics) may be impaired with jeopardy of the whole pharmaceutical endeavor.

What is needed is a PDT reactive composition that possesses the ability to overcome the barriers associated with the epithelium and cell membrane.

### SUMMARY OF THE INVENTION

The present invention discloses the local use of a modified photosensitive molecule for the purpose of photodynamic treatment of tissue maladies, including but not limited to, neoplasms and hyperplasias.

In embodiments, a method of photodynamic disruption of cells is disclosed including contacting a cell with a composition comprising a photosensitive structure as set forth in Formula (I):

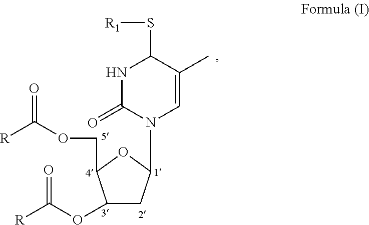

where R is an alkyl group or an alkylene group between 6 and 20 carbon atoms in length, an hydroxylated alkyl group or hydroxylated alkylene group between 6 and 20 carbon atoms in length, a lipoamino acid group, or a sugar acid group, where R₁ is an alkyl group or an alkylene group between 1 and 15 carbon atoms in length, and where the structure passes through the cell membrane and into the cell interior; and applying light on the cell to cause a disruption of the cell by a photodynamic reaction of the photosensitive structure within the cell.

In one aspect, the contacting step includes disposing of the composition proximate to the cell. In a related aspect, the proximate disposing includes an intravenous injection, a subcutaneous injection, an intratumoral injection, and a topical application.

In another aspect, the cell is actively proliferating. In a related aspect, the cell is a skin cell, where the skin cell is neoplastic. In a further related aspect, the neoplastic skin cell includes a head and neck cancer cell, psoriatic cell, actinic keratotic cell, and keloid cell. In another related aspect, the cell is cancer cell of the stomach, colon, or bladder.

In one aspect, the step of applying light occurs for a period of between about 5 seconds to about 1 hour. In another aspect, the wavelength of light applied ranges from about 400 nm to 315 nm at a dosage ranging from about 1 kJ/m² to about 50 kJ/m², In one aspect, the photosensitive structure is present at a concentration range of between about 3 μg/ml to about 500 μg/ml of the composition.

In another aspect, the cell includes eucaryotic cells, prokaryotic cells, obligate intracellular bacteria cells, bacteria cells, virally infected cells, and cancer cells.

In another embodiment, a method of treating an epithelial hyperplasia is disclosed, including administering a pharmaceutically effective amount of a composition containing a photosensitive structure to a subject in need thereof, where the structure is as set forth in Formula (II):

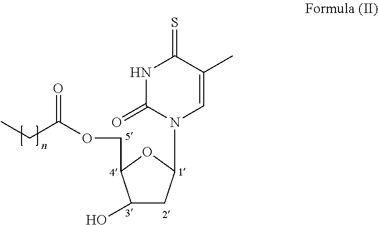

where n is 14, and where the structure passes through a cell membrane and into a cell interior; and applying light on the subject, where the light induces a photodynamic reaction of the photosensitive structure within cells of the epithelial hyperplasia.

In one aspect, the method further comprises pre-treating the epithelial hyperplasia with an aprotic solvent and a physiological buffer. In a related aspect, the aprotic solvent is DMSO and the physiological buffer is phosphate buffered saline or HEPES.

In one embodiment, a kit is disclosed including a composition containing a photosensitive structure as set forth in Formula (I):

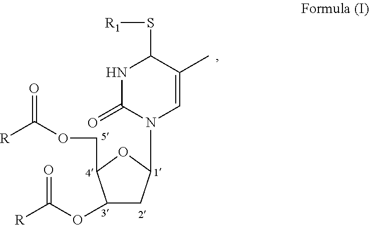

where R is an alkyl group or an alkylene group between 6 and 20 carbon atoms in length, an hydroxylated alkyl group or hydroxylated alkylene group between 6 and 20 carbon atoms in length, a lipoamino acid group, or a sugar acid group, and where R₁ is an alkyl group or an alkylene group having 0 to 15 carbon atoms; a container; optionally one or more buffers and solvents; a label; and instructions on how to apply to the composition to cells.

In a related aspect, the kit further comprises a light source which is adapted to apply a wavelength of light in the range from about 400 nm to about 315 nm at a dosage ranging from about 1 kJ/m² to about 50 kJ/m².

In another embodiment, a use of a composition containing a photosensitive structure is disclosed, where the structure is as set forth in Formula (II):

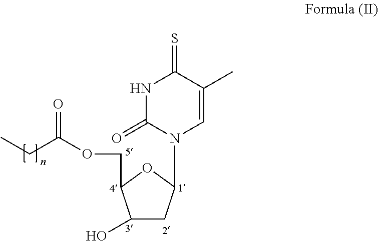

where n is 14, and wherein said structure passes through a cell membrane and into a cell interior; for the production of a medicament for the treatment of a neoplasm in a subject in need thereof, where when light is applied on the subject, the light induces a photodynamic reaction of the photosensitive structure within cells of the neoplasm.

In a related aspect, the neoplasm is an epithelial hyperplasia.

### BRIEF DESCRIPTION OF THE DRAWINGS

FIG. 1 shows an illustration of the different layers comprising the skin

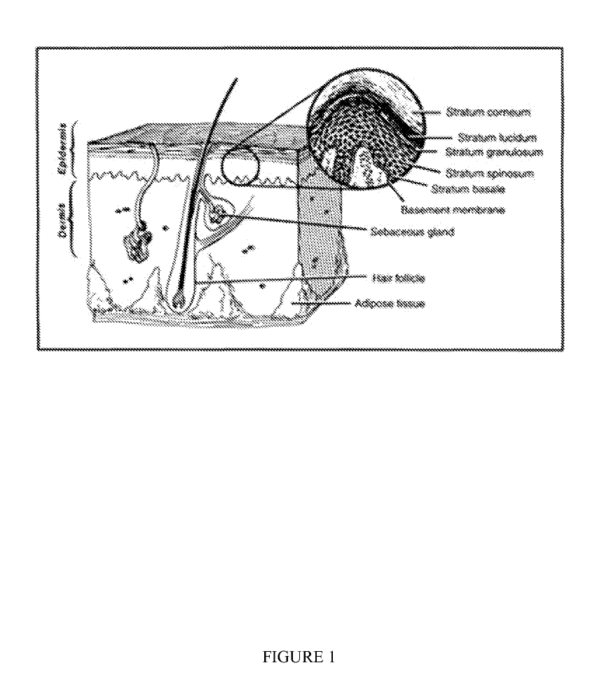

FIG. 2 shows the structures of thymidine (T) and 4-thiothymidine (4-TT).

FIG. 3 shows the structure of substituted 4-thiothymidine (4-TT, Formula (I)).

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “an agent” includes one or more agents, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term and “substantially” and “significantly” will mean plus or minus >10% of the particular term.

As used herein “photosensitive structure” means a molecule or compound which is responsive or reactive to light or other radiant energy.

As used herein “photodynamic” means enhancing the effects of or inducing a toxic reaction to light (e.g., use of UV light to produce such an effect)

As used herein “neoplastic”, including grammatical variations thereof, means an abnormal growth of tissues in an animal.

As used herein “epithelial hyperplasia” means alterations in structure, produced by proliferation of cellular elements of the cellular covering of internal and external body surfaces, including the lining of vessels and small cavities.

As used herein “aprotic solvent” means a solvent that does not accept or yield protons (e.g., DMSO is an aprotic solvent).

As used herein “physiological buffer” means a combination of salts in solution which help to maintain the pH, osmolarity, and ion concentrations which match those of the human body.

As used herein “lipoamino acid” means any of several classes of lipids, containing amino acid residues, with or without glycerol, and/or fatty acid residues, but lacking a phosphate group.

As used herein “sugar acid” means a monosaccharide that contains a carbonyl group, including, but not limited to, aldonic acids, ulosonic acids, cronic acids, and aldaric acids.

By “topical formulation” it is meant that the dermatological agent is present in a form that is capable of application to the surface of the skin and is able to be absorbed through the skin. Such topical formulations of dermatological agents are typically in the form of a cream, lotion, ointment, gel, solution, foam, powder, and the like. The concentration of the dermatological agent will depend on the particular agent, the particular disease disorder, the host, the site of application, and the like.

Dosage forms for topical applications may include solutions, nasal sprays, lotions, ointments, creams, gels, suppositories, sprays, aerosols as well as devices such as skin patches, bandages and dressings containing a composition according to the invention. Typical conventional pharmaceutical carriers which make up the foregoing dosage forms include water, acetone, isopropylalcohol, ethylalcohol, polyvinylpyrrolidone, propylene glycol, fragrances, gel-producing materials, mineral oil, stearyl alcohol, steric acid, spermaceti, sorbitan monoleate, “Polysorbates”, “Tweens”, and the like.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “pharmaceutically acceptable carrier” means a chemical composition with which the active ingredient may be combined and which, following the combination, can be used to administer the active ingredient to a subject.

The term “pharmaceutical composition” refers to a mixture of a compound with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary, and topical administration.

### PDT

Photodynamic therapy (PDT) is a promising non-surgical technique that involves the systemic or topical application of a photosensitizing drug that is preferentially retained in tumors, and with exposure to light of the correct wavelength, results in selective destruction of cancerous cells. Initial studies with PDT show good cure rates and excellent cosmetic results for superficial tumors.

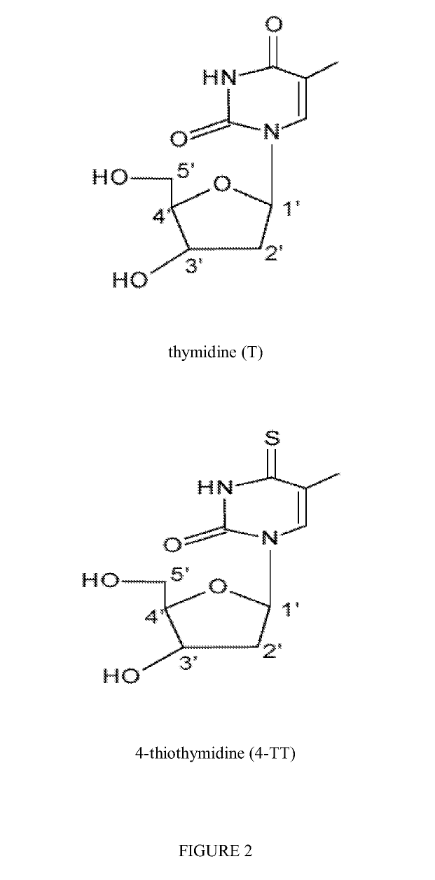

The present disclosure describes the local use of a modified novel molecule for the purpose of photodynamic treatment of tissue hyperplasias. The molecule is called 4-thiothymidine (4-TT) and is a derivative of the nucleotide thymidine, present in DNA (FIG. 2).

Thymidine is a pyrimidine nucleotide, one of the four building blocks of DNA. As such, it is needed by all cells in a state of proliferation in order to replicate their DNA. Upon exposure to UV-B, a harmful form of ultraviolet radiation, thymidine undergoes a photochemical reaction which leads to its dimerization to form thymidine dimers, a potentially DNA damaging species. This is one reason why the skin needs protection from UV-B, which is present in small amounts in sunlight. On the contrary, the UV-A fraction of sunlight is harmless to thymidine and DNA.

Recent research by P. Karran and colleagues (Massey A, Xu Y Z, Karran P., Curr Biol. 2001 Jul. 24; 11(14):1142-6) has illustrated the potential for the use of a novel thymidine derivative, 4 thiothymidine, in the fight against cancer. This modified thymidine molecule displays a shift in its absorbance peak from 260 nm (UV-B) to 335 nm (UV-A). Excitation of the molecule at this wavelength induces a photochemical reaction which results in toxicity to the cells which have incorporated the drug. Exposure to drug alone or to UV-A alone does not result in appreciable toxicity. The molecule is therefore an excellent candidate for photodynamic therapy. In particular, its natural tendency as a nucleotide to concentrate in proliferating cells' DNA provides it with an advantage over other PDT drugs. Moreover, the fact that UV-A radiation is less common than red light and requires direct exposure to sunlight, makes the issue of side effects and patient protection even less relevant. The precautions associated with the use of PHOTOFRIN would therefore not be applicable to this new drug.

In addition to the base compound 4 thiothymidine, modifications of the molecule may be devised which allow similar or better performance by enhancing delivery of the compound to target tissues. In accordance to the present disclosure, the active ingredient 4-TT may be administered to the patient lesion area locally by means of a penetrating formulation.

All human epithelia, and the skin in particular, exhibit some kind of barrier effect to prevent indiscriminate crossing of compounds. The skin is particularly apt to this purpose by means of the so called cornified layer which is the thin but very impermeable outermost coating of the skin, made of dead cells cemented together by keratins and lipids. Crossing this barrier for the purpose of drug delivery is a formidable challenge. A considerable amount of knowledge exists in the art concerning manners to overcome the cornified layer barrier. For example, it has been observed that pre-treatment of the skin with solvents, moisturizers of specific wetting compounds (e.g., aprotic solvents such as acetone, Azone, dimethylsulfoxide, 1-methyl-2-pyrrolidone, decylmethylsulfoxide, polyethylene glycol) facilitates subsequent penetration of applied formulations.

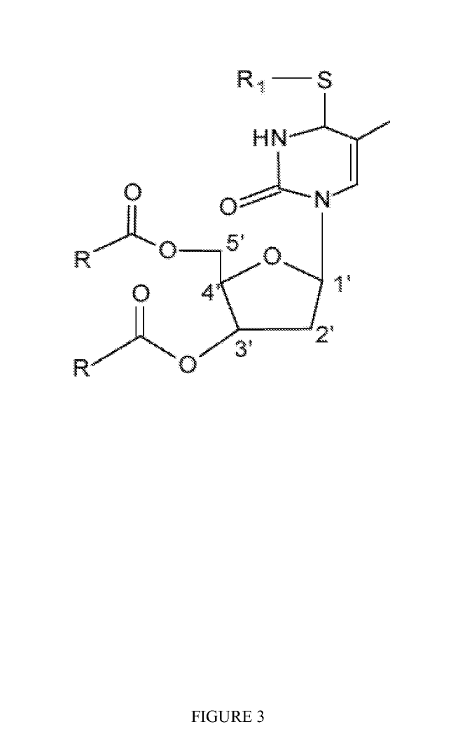

In embodiments, a strategy to improve drug bioavailability at the target site is the chemical derivatization of the drug itself with substituents designed to alter the physico-chemical characteristics of the parent compound to make it more apt at penetrating the biological barrier of application, be it the skin, oral/gastric mucosa, bronchial mucosa, bladder lining (henceforth called the Barrier). Said substituents can be attached to the hydroxyl groups on the sugar part of the molecule (e.g., the 3′, 5′ positions) or to the sulphur atom on the pyrimidine ring (4 position) (FIG. 3). The basic requirements for any such substituent is the prompt cleavage they would undergo once inside the target cells to release the original active drug 4-TT. This can easily be accomplished by attaching the modifying groups to the hydroxyl groups by means of ester bonds because cells contain non-specific esterase enzymes which are able to readily cleave such ester bonds.

The current literature reports a vast repertoire of such molecules apt at modifying the chemical nature of a drug and producing a prodrug, most notably in order to increase a drug's hydrophobicity and allow passage through the skin or other epithelia. The prodrug is then hydrolysed back to the plain drug by cellular metabolism.

In embodiments, such modifying molecules include alkanic or alkenic acid groups or derivatives thereof: these are linear or branched chain hydrocarbons with a length from 6 to 20 carbon atoms and with possible unsaturated moieties and hydroxyl substitutions. Examples include, but are not restricted to, capric acid, octanoic acid, oleic acid, butyric acid, valeric acid, caproic acid, caprylic acid, lauric acid, myristic acid, palmitic acid, ricinoleic and stearic acid.

In embodiments, such modifying molecules include amino modified hydrocarbons: i.e., lipoamino acids. These are constituted of a linear alkyl or alkenyl acid chains conjugated by an amide bond with an amino acid such as proline, lysine etc., whose terminal carboxylic acid group can then be conjugated to 4-TT. In embodiments, amino acids include, but are not limited to, proline, valine, isoleucine, and arginine.

In embodiments, such modifying molecules include sugar acids, such as glutaric acid, mannosic acid, and the like.

In embodiment, 4-S-sulfenylalkyl (—SR) groups on the 4-S atom of 4-TT are also included as substituents of the modified molecule.

Accordingly, the present invention encompasses a medicine for photodynamic therapy which contains the compounds of the present disclosure. Further, a method for treating cancer by administering the compound of the present disclosure into a subject, particularly, a method for treating cancer by photodynamic therapy is also encompassed in the present disclosure. The administration of the medicine or the compound into a living organism may be carried out by injection via various paths, but is not limited in any particular manner. Further, doses of the medicine or the compound may be appropriately designed by a skilled person in the related art, as needed.

Prior to application of the formulation the barrier may be treated with compounds which are known to facilitate subsequent penetration of formulations such as AZONE™ (Ziolkowski P, et al., J Environ Pathol Toxicol Oncol. 2006; 25(1-2):403-9), or decylmethylsulphoxide (Choi H K, Amidon G L, Flynn G L., J Invest Dermatol. 1991 June; 96(6):822-6). The formulation itself may be applied directly, through the use of occlusive dressing or in the form of patch. Alternatively, it may be applied by means of an endoscopic probe or catheter.

Following application, a lag time may be observed to allow metabolism of the drug into cells and their DNA. In embodiments, such lag time may be between about 0.1 to about 0.5 hrs, about 1 hr to about 5 hrs, about 5 hrs to about 10 hrs, or between about 12 hrs to about 48 hrs. Following this lag time, a UV-A radiation of appropriate penetrating intensity and energy is applied.

A light source is utilized to practice embodiments of the present invention. The light source may be laser light source, a high intensity flash lamp, or other illumination sources as appreciated by those skilled in the relevant arts. A broad spectrum light source may be utilized, however a narrow spectrum light source is one preferred light source. The light source may be selected with reference to the specific photosensitive material, as photosensitive materials may have an associated range of photoactivation.

In embodiments, a laser light source may be used to practice the present methods. A variety of laser light sources are currently available, and the selection of a particular laser light source for implementing the PDT would readily be appreciated by those skilled in the relevant arts. A hand manipulable light wand or fiber optic device may be used to illuminate tissue within a living body. Such fiber optic devices may include a disposable fiber optic guide provided in kit form with a solution containing a photosensitive material and optionally one or more solvents or buffers. Other potential light devices for use in accordance with the present disclosure include the devices disclosed U.S. Pat. No. 6,159,236 and U.S. Pat. No. 6,048,359, both incorporated in their entireties by reference herein. The laser source may be selected with regard to the choice of wavelength, beam diameter, exposure time and sensitivity of the cellular and/or acellular organisms to the laser/photosensitizer/surfactant combination. In embodiments, the light source is utilized for a period of time necessary to affect a photodynamic response. The period of time for photodynamic activation of the photosensitive material may be between 5 seconds and 1 hour. In embodiments, the period of time for light illumination is between 2 and 20 minutes.

Repeat administrations of a treatment protocol may also be necessary or desired, including repeat administrations of solvents/buffers and photosensitive materials and light activation. The repeat administrations may include different solvents/buffers and/or photosensitive materials than previously administered. Repeat administrations of the treatment protocol may continue for a period of time.

Additional aspects of the present disclosure include administration or delivery approaches of the photosensitive material and solvent/buffer. In one embodiment, the photosensitive material and the solvent are provided in a combined solution and topically applied to the cell site. In other embodiments, the photosensitive material may be applied or delivered or dispensed to a tissue site before, during, or after the application or delivery of the solvent through known delivery/administration approaches. In one embodiment, a topical solvent application would precede a topical photosensitive material application by 1-30 minutes.

Additional aspects of the present disclosure further include combinations of different photosensitive materials during a treatment protocol. In embodiments, a particular combination of a photosensitizer would be dispensed to the tissue site in association with a first photodynamic illumination of the tissue site. After a period of time, another different particular photosensitizer would be dispensed to the tissue site in association with a second photodynamic illumination of the tissue site.

In embodiments, the wavelength of the applied light covers the absorption maximum of 4-TT which is about 335 nm. For this purpose any suitable UV-visible light source may be used with emission spectra from 300 nm to 600 nm or 315 nm to 400 nm. The source emission spectrum must cut off abruptly under 300 nm at most in order not to include harmful UV-B radiation.

The outermost cells in the Barrier will be most affected and are expected to die of cellular apoptosis within 24 hours. Since the depth of drug penetration and incorporation is expected to exceed that of UV radiation penetration, one round of irradiation will probably not cover the whole lesion and therefore repeated applications are allowed; these are made possible by the known safety of UV-A radiation.

In the case of the digestive tract the employment of photodynamic therapy is all the more desirable since classical chemotherapy cannot be administered topically as some absorption through the walls of the intestine is unavoidable. Particularly, in the case of the mouth the constant flux of saliva would rapidly cause ingestion and absorption in the bloodstream of any classical chemotherapeutic. The compositions described in the present disclosure are aimed at topical delivery of the drug.

In addition to the above, the present compounds are used as photosensitizing drugs for PDT in veterinary applications, for example in treatment of cancers such as ear cancer in cats, as antifungal, antibacterial and antiviral treatments, for sterilization of wounds in animals and for ophthalmological treatments in animals.

The use of the compounds of Formula (I)

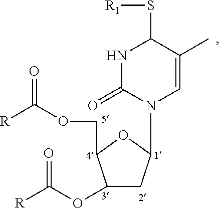

where R is an alkyl group or an alkylene group between 6 and 20 carbon atoms in length, an hydroxylated alkyl group or hydroxylated alkylene group between 6 and 20 carbon atoms in length, a lipoamino acid group, or a sugar acid group, where R₁ is an alkyl group or an alkylene group between 1 and 15 carbon atoms in length, may be used in treatments of localized and/or early cancer and/or pre-cancerous lesions in humans and in animals; or in the treatment and/or prevention of infections in wounds or skin in humans and animals.

According to a further feature of the present disclosure the present compounds may be used as photo activated antimicrobial, antifungal and antiviral agents for sterilization of surfaces and fluids, for example they may be used to sterilize surgical implants and stents, particularly where these are coated or impregnated, to sterilize textiles such as bandages and dressings, IV lines and catheters, for sterilization of water, air, blood, blood products, and food and food packaging to prevent transfer of infection, and for general household, hospital and office cleaning. The compounds may be used to sterilize surgical implants and stents, particularly where these are coated or impregnated, to sterilize textiles such as bandages and dressings, IV lines and catheters, for sterilization of water, air, and food and food packaging to prevent transfer of infection, and for general household, hospital and office cleaning. The compounds may be applied to or contacted with the surfaces and fluids and activating the compound by exposure to light. Additionally the surface to be sterilized may be immersed in a mixture or solution of the compound or the fluid to be sterilized may be mixed with the compound or a solution or mixture containing the compound.

Where the compounds of the present invention are used as PDT agents for mammalian cells and tumors they may be administered using the above described compositions in a variety of ways, such as systemically or locally and may be used alone or as components or mixtures with other components and drugs. Where administered systemically the compounds may be delivered for example intravenously, orally, sub-cutaneously, intramuscularly, directly into affected tissues and organs, intraperitoneally, directly into tumors (intratumorally), intradermally or via an implant. Where administered locally or topically the compounds may be delivered via a variety of means for example via a spray, lotion, suspension, emulsion, gel, ointment, salves, sticks, soaps, liquid aerosols, powder aerosols, drops or paste.

According to a further feature of the present invention there is provided a method of treatment of microbial infections, burn wounds and other lesions and of dental bacterial disease, the method comprising systemic administration or applying to the area to be treated (for example by a spray, lotion, suspension, emulsion, ointment, gel or paste) a therapeutically effective amount of a compound of the present disclosure and exposing said area to light to render active said compound.

The compounds of the present invention are particularly useful as photosensitizing drugs for PDT of conditions where treatment requires removal, deactivation or killing of unwanted tissue or cells such as cancer, precancerous disease, ophthalmic disease, vascular disease, autoimmune disease, and proliferative conditions of the skin and other organs. Specific and unpredicted advantages of these materials relate to their ability to be photoactive against target tissues at different times after systemic administration (depending upon the particular sensitizer used) and therefore their ability to be targeted directly for example to the vasculature or tumor cells. They also have a low tendency to sensitize skin to ambient light when administered systemically and a low tendency to color skin.

In embodiments, a method is disclosed of treatment for cancer and other human or animal diseases through systemic or local administration of the photo sensitizer, followed by application of light of an appropriate dose and wavelength or wavelength range.

For the present compounds activation is by light, including white light, of an appropriate wavelength (e.g., UVA; 400-315 nm, 3.10-3.94 ev; long wave, black light).

The light source may be any appropriate light source such as a laser, laser diode or non-coherent light source. The light dose administered during PDT can vary but preferably is from 1 to 200 J/cm², more preferably from 20 to 100 J/cm².

Light exposure may be given at any time after a drug is initially administered or up to 48 hours after drug administration and the time may be tailored according to the condition being treated, the method of drug delivery and the specific compound of Formula (I) used. Light exposure may be given at any time after a drug is initially administered up to 3 hours, in embodiments, from the time after a drug is initially administered up to 1 hour, in embodiments, up to 10 minutes. In embodiments, light exposure is given within 1 minute after a drug is initially administered. In embodiments, light exposure is given at the point of drug administration.

Increased intensity of the light dose generally reduces exposure times.

In embodiments, exposure to light is localized to the area/region to be treated, and where tumors are being treated, in embodiment, localized to the tumor itself (e.g., intratumoral).

The dose rates of the compounds of Formula I for intravenous administration to humans for oncology treatments may be in the range of about 0.01 to about 10 μmol (micromole)/kg, in the range of about 0.1 to about 2.0 μmol (micromole)/kg. In embodiments, to achieve a dose of about 2 mol (micromole)/kg in a 70 kg patient may require injection of about 70 ml of a 2 mM solution, or about 5 ml at a concentration of 27 mM (16 mg/ml) or about 2.8 ml of a 50 mM solution. Typical injections volumes may be in the range 0.1 to 100 ml, or from about 5 to about 50 ml.

According to a further feature of the present disclosure there is provided a method of prevention of microbial infections, for example in wounds, surgical incisions, burn wounds, and other lesions and of dental bacterial disease, the method comprising systemic administration or applying to the area to be treated (for example by a spray, lotion, suspension, emulsion, ointment, gel or paste) a therapeutically effective amount of a compound of the present disclosure and exposing said area to light to render active said compound. The compounds of Formula I may be applied to prevent infection at any stage including wound contamination, where non-replicating organisms are present in a wound; wound colonization where replicating microorganisms are present in a wound; and wound infection where replicating microorganisms are present that cause injury to the host. When there are >10⁵ CFU/g tissue, it is more likely that sepsis will develop.

The concentration used for bacterial cell kill in vitro may be in the range from about 0.1 to about 100 μM, in embodiments from about 1 to about 50 μM, in embodiments, from about 5 to about 20 μM, in embodiments about 10 μM.

### Pharmaceutical Composition/Formulation

In embodiments, the compounds described herein are formulated into pharmaceutical compositions. In embodiments, pharmaceutical compositions are formulated in a conventional manner using one or more physiologically acceptable carriers comprising excipients and auxiliaries which facilitate processing of the active compounds into preparations which may be used pharmaceutically. Proper formulation is dependent upon the route of administration chosen. Any pharmaceutically acceptable techniques, carriers, and excipients may be used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiological buffers; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The active ingredient combinations of the invention may be provided as components of a pharmaceutical pack, referred to herein as a “kit”. The components (e.g., a modified 4-TT and additional ingredients) may be formulated together or separately.

The following examples are intended to illustrate but not limit the invention.

### Examples

### Example 1

A patient suffering from a basal cell carcinoma (BCC) lesion on the arm is treated in the following way. The lesion is cleaned, then pre-treated with acetone and DMSO for 10 min. Following this a gel consisting of 10 μM 4-TT-5′-palmitate, 40% DMSO in saline buffer. The lesion is dressed with surgical membrane and left untouched for 4 hours. After this period the dressing is removed and the lesion cleaned and dressed normally. 20 hours later the lesion is irradiated with a UV-A lamp with an emission centered at 350 nm, for a period of 10 min and a total energy of 10 kJ/m². The irradiation is repeated for one week, following which the whole treatment is repeated three times. Regression of the BCC is then assessed by biopsy and photography.

### Example 2

A patient suffering from bladder cancer has the lesion directly covered, by means of a probe, with a solution of 50 μM 4-TT-5′-valinate in 20% DMSO, 10% PEG and 70% HEPES buffer. The application repeated after four hours, and once more after that. The following day, at 24 hours from the last application, UV-A light is shined on the lesion with an emission maximum of 350 nm and 20 min application, for a total energy of 20 kJ/m². The irradiation is repeated for 20 days and regression of the lesion monitored photographically.

Although the invention has been described with reference to the above examples, it will be understood that modifications and variations are encompassed within the spirit and scope of the invention. Accordingly, the invention is limited only by the following claims.

All references disclosed herein are incorporated by reference in their entireties.

1. A method of photodynamic disruption of cells comprising:
contacting a cell with a composition comprising a photosensitive structure as set forth in Formula (I): 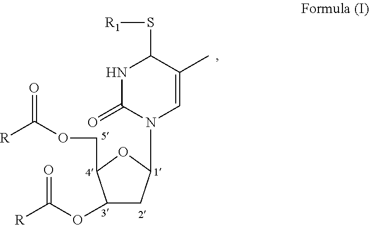
wherein R is an alkyl group or an alkylene group between 6 and 20 carbon atoms in length, an hydroxylated alkyl group or hydroxylated alkylene group between 6 and 20 carbon atoms in length, a lipoamino acid group, or a sugar acid group,
wherein R₁ is an alkyl group or an alkylene group between 1 and 15 carbon atoms in length, and wherein said structure passes through the cell membrane and into the cell interior; and
applying light on said cell to cause a disruption of the cell by a photodynamic reaction of said photosensitive structure within the cell.

2. The method of claim 1, wherein said contacting step comprises disposing of the composition proximate to the cell.

3. The method of claim 2, wherein the proximate disposing is selected from the group consisting of an intravenous injection, a subcutaneous injection, intratumoral injection, and a topical application.

4. The method of claim 1, wherein said cell is actively proliferating.

5. The method of claim 4, wherein said cell is a skin cell, and wherein said skin cell is neoplastic.

6. The method of claim 5, wherein said neoplastic skin cell is selected from the group consisting, of head and neck cancer cell, psoriatic cell, actinic keratotic cell, and keloid cell.

7. The method of claim 4, wherein the cell is cancer cell of the stomach, colon, or bladder.

8. The method of claim 1, wherein the step of applying light occurs for a period of between about 5 seconds to about 1 hour.

9. The method of claim 1, wherein the wavelength of light applied ranges from about 400 nm to 315 nm at a dosage ranging from about 1 kJ/m² to about 50 kJ/m², and wherein the photosensitive structure is present at a concentration range of about 3 μg/ml to about 500 μg/ml of said composition.

10. The method of claim 1, wherein the cell is selected from the group consisting of eukaryotic cells, prokaryotic cells intracellular bacteria, bacteria, virally infected cells, and cancer cells.

11. A method of treating an epithelial hyperplasia comprising:
administering a pharmaceutically effective amount of a composition containing a photosensitive structure w a subject in need thereof, wherein said structure is as set forth in Formula (II): 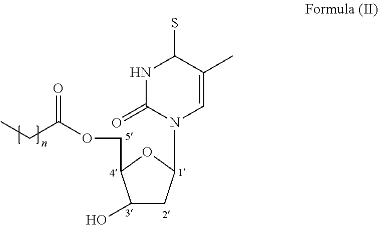
wherein n is 14, and wherein said structure passes through a cell membrane and into cell interior of a cell of the epithelial hyperplasia; and
applying light on said subject,
wherein said light induces a photodynamic reaction of said photosensitive structure within cells of the epithelial hyperplasia.

12. The method of claim 11, further comprising pre-treating the epithelia hyperplasia with an aprotic solvent and a physiological buffer.

13. The method of claim 12, wherein the aprotic solvent is DMSO and the physiological buffer is phosphate buffered saline or HEPES.

14. The method of claim 11, wherein the epithelial hyperplasia is head and neck cancer, basal cell carcinoma, psoriasis; actinic keratosis, or keloids.

15. The method of claim 11, wherein the wavelength of light applied ranges from about 400 nm to 315 nm at a dosage ranging from about 1 kJ/m² to about 50 kJ/m².

16. The method of claim 11, wherein the photosensitive structure is present at a concentration range of about 3 μg/ml to about 500 μg/ml of said composition.

17. The method of claim 11, wherein said administering step comprises disposing of the composition proximate to the cell, and wherein said proximate disposing is selected from the group consisting of an intravenous injection, as subcutaneous injection, intratumoral injection, and a topical application.

18. The method of claim 11, wherein the step of applying light occurs for a period of between about 5 seconds to about 1 hour.

19. A kit comprising:
(a) a composition comprising a photosensitive structure as set forth in Formula (I): 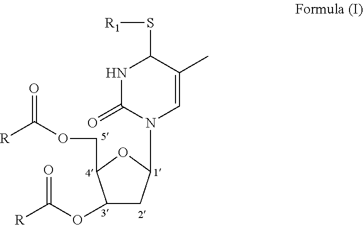
wherein R is an alkyl group or an alkylene group between 6 and 20 carbon atoms in length, an hydroxylated alkyl group or hydroxylated alkylene group between 6 and 20 carbon atoms in length, a lipoamino acid group, or a sugar acid group, and
wherein R₁ is an alkyl group or an alkylene group having 0 to 15 carbon atoms;
(b) a container;
(c) optionally one or more buffers and solvents;
(d) a label; and
(e) instructions on how to apply to the composition to cells.

20. The kit of claim 19, further comprising a light source which is adapted to apply a wavelength of light in the range from about 400 nm to about 315 nm at a dosage ranging from about 1 kJ/m² to about 50 kJ/m².

21.-27. (canceled)
